(19) United States
(12) Patent Application Publication
(10) Pub. No.: US 2021/0315718 A1
(43) Pub. Date: Oct. 14, 2021

(54) POSITIONING DEVICE FOR STENT

(71) Applicants: Po Jen Ko, Taoyuan City (TW); Ta-Wei Su, Tainan City (TW); I-Hao Su, Taoyuan City (TW)

(72) Inventors: Po Jen Ko, Taoyuan City (TW); Ta-Wei Su, Tainan City (TW); I-Hao Su, Taoyuan City (TW)

(73) Assignee: Taiwan Association of Vascular and Access Health, Taoyuan City (TW)

(21) Appl. No.: 16/847,813

(22) Filed: Apr. 14, 2020

(51) Int. Cl. A61F 2/95; A61F 2/06

(52) U.S. Cl. CPC A61F 2/95; A61F 2/06; A61F 2002/045; A61F 2002/9522; A61F 2210/009; A61F 2002/9517

(57) ABSTRACT

A positioning device for a stent includes a positioning member including a handle and an extension secured to the handle wherein the handle and the extension are coaxially connected, and an open end of the extension is provided with a hollow first magnetic member; and a stent inserted into a blood vessel and including a first stent member inserted into an artificial blood vessel, the first stent member being separated into two second stent members inserted into the artificial blood vessel wherein a diameter of the first stent member is greater than that of each second stent member, and a diameter of each second stent member is greater than that of the extension, the stent further comprising at least one second magnetic member on the first stent member proximate an open end thereof, the second magnetic member being configured to attach to the first magnetic member.

### BACKGROUND OF THE INVENTION

### 1. Field of the Invention

The invention relates to positioning devices and more particularly to a positioning device for a stent in which a positioning member is placed in a stent which is inserted into a blood vessel, the positioning member has a first magnetic member, and the stent has second magnetic members adapted to attach to the first magnetic member so that the positioning member and the stent can be releasably fastened together, the positioning member is capable of locating corresponding screws in the stent, and the stent is secured to an aorta and artery of abdomen.

### 2. Description of Related Art

In the procedure of treating aortic aneurysm, a metal guide wire is inserted into the aorta. Next, aortography is performed to identify the aneurysm of aorta and ascertain both length and diameter of the aortic aneurysm. These data are used to select an appropriate stent. Next, the guide wire is advanced through the blood vessel. Next, the stent passes over the guide wire until it reaches a predetermined position in the blood vessel. Finally, both a sheath of the stent and the guide wire are removed. As a result, the stent is expanded so as to keep the blood vessel open. The endovascular treatment of aortic aneurysm has the following advantages in comparison with the conventional incision through the abdomen based on clinical study: short operation time, a small quantity of blood being lost in operation, and quick recovery time after operation.

It is typical of using screws to secure the stent to the blood vessel for preventing the stent from being disengaged from the blood vessel. However, it is difficult of positioning the positioning member to a predetermined position of the stent by screwing.

Thus, the need for improvement still exists.

### SUMMARY OF THE INVENTION

It is therefore one object of the invention to provide a positioning device for a stent by attracting one magnetic member to another magnetic member so that it is possible of positioning a positioning member to a predetermined position of the stent.

For achieving above and other objects, the invention provides a positioning member including a handle and an extension secured to the handle wherein the handle and the extension are coaxially connected, and an open end of the extension is provided with a hollow first magnetic member; and a stent inserted into a blood vessel and including a first stent member inserted into an artificial blood vessel, the first stent member being separated into two second stent members inserted into the artificial blood vessel wherein a diameter of the first stent member is greater than that of each second stent member, and a diameter of each second stent member is greater than that of the extension, the stent further comprising at least one second magnetic member on the first stent member proximate an open end thereof, the second magnetic member being configured to attach to the first magnetic member.

By utilizing the invention, the second magnetic member is attached to the first magnetic member due to magnetic field. The open end of the extension can be automatically attached to the second magnetic member. A screw is provided in the positioning member. The screw moves along an axis of the extension until reaches an end of the first magnetic member. The second magnetic members thus attach to the first magnetic member. The screw is secured to the second magnetic member. The stent is secured to an artery of abdomen. This is a quick fastening process.

The above and other objects, features and advantages of the invention will become apparent from the following detailed description taken with the accompanying drawings.

### BRIEF DESCRIPTION OF THE DRAWINGS

FIG. 1 is a perspective view of a positioning member according to the invention;

FIG. 2 is a perspective view of a stent according to a first preferred embodiment of the invention;

FIG. 3 is a perspective view of a stent according to a second preferred embodiment of the invention;

FIG. 4 is a perspective view of a stent according to a third preferred embodiment of the invention;

FIG. 5 is a perspective view of a stent according to a fourth preferred embodiment of the invention;

FIG. 6 is a sectional view showing the stent placed in an artery of abdomen, the extension passing through the stent, and the first magnetic member attached to a predetermined position proximate the second magnetic member according to the invention; and

FIG. 7 is a sectional view of a plurality of screws secured to the second magnetic members according to the invention.

### DETAILED DESCRIPTION OF THE INVENTION

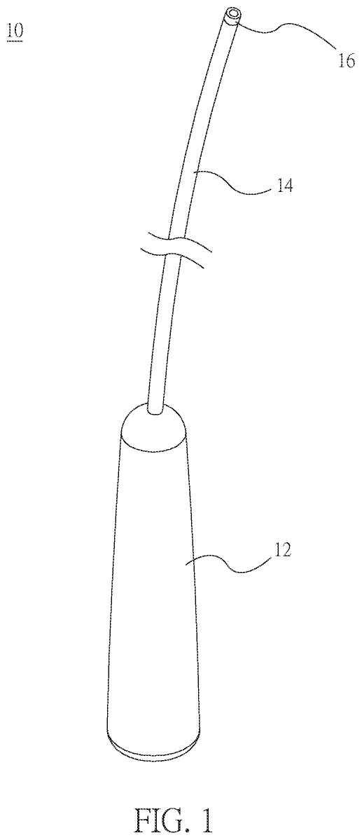

Referring to FIG. 1, a positioning device for stent in accordance with the invention comprises a positioning member 10 including a handle 12 and an extension 14 secured to the handle 12. The handle 12 and the extension 14 are coaxially connected. An open end of the extension 14 is provided with a hollow first magnetic member 16.

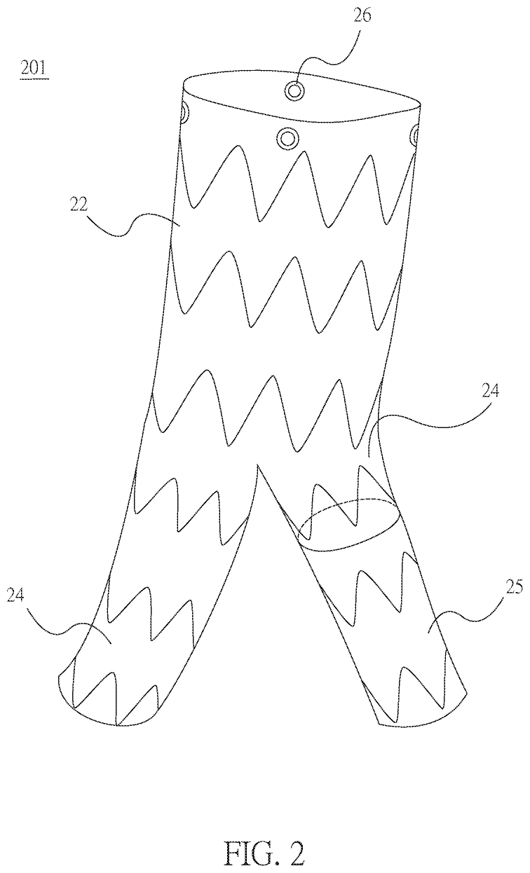

Referring to FIG. 2, a stent 201 in accordance with a first preferred embodiment of the invention is shown. The stent 201 is inserted into a blood vessel and is implemented as including a first stent member 22 inserted into an artificial blood vessel. The first stent member 22 is separated into two second stent members 24 inserted into the artificial blood vessel. As shown in the right side of FIG. 2, a third stent 25 is extended from the second stent member 24 into the artificial blood vessel. A diameter of the first stent member 22 is greater than that of each second stent member 24. A diameter of each second stent member 24 is greater than that of the extension 14. A plurality of hollow circular second magnetic members 26 are spaced around the first stent member 22 proximate an open end of the first stent member 22. The second magnetic members 26 are configured to attach to the first magnetic member 16 due to magnetic field. The first stent member 22 and the second stent members 24 are formed integrally.

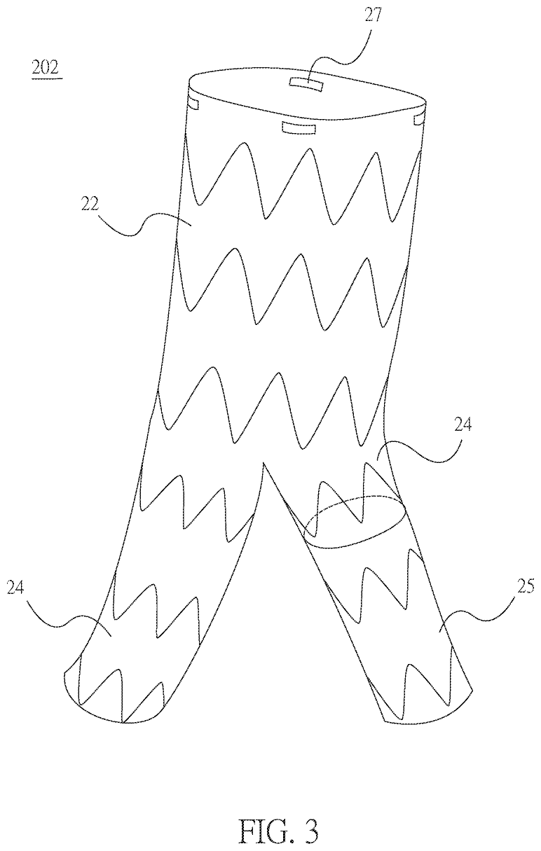

Referring to FIG. 3, a stent 202 in accordance with a second preferred embodiment of the invention is shown. The characteristics of the stent 202 of the second preferred embodiment are substantially the same as the stent 201 of the first preferred embodiment except the following: a plurality of second magnetic members 27 are solid rectangular.

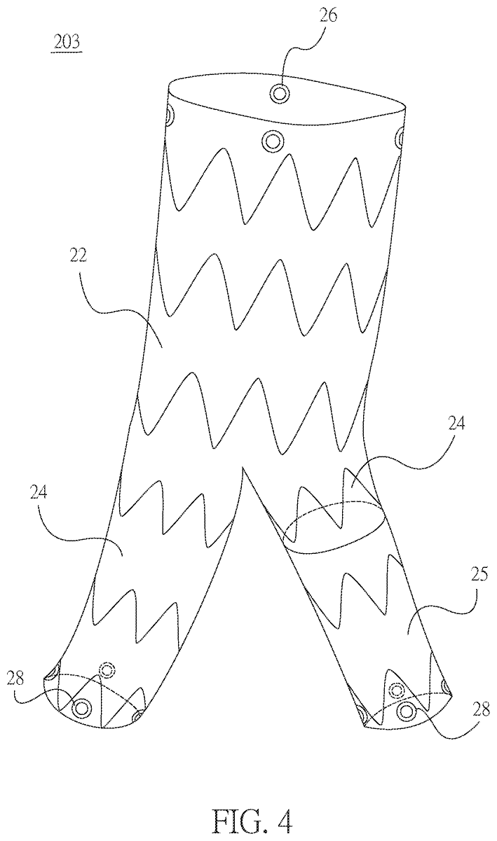

Referring to FIG. 4, a stent 203 in accordance with a third preferred embodiment of the invention is shown. The characteristics of the stent 203 of the third preferred embodiment are described below. The stent 203 is inserted into a blood vessel and is implemented as including a first stent member 22 inserted into an artificial blood vessel. The first stent member 22 is separated into two second stent members 24 inserted into the artificial blood vessel. A third stent 25 into the artificial blood vessel is extended from either one of the second stent members 24. As shown in FIG. 4, the third stent 25 is extended from the right second stent member 24 and inserted into the artificial blood vessel. A plurality of third magnetic members 28 are spaced around the third stent 25 proximate an open end of the third stent 25. Further, a plurality of third magnetic members 28 are spaced around the second stent member 24 proximate an open end of the second stent member 24.

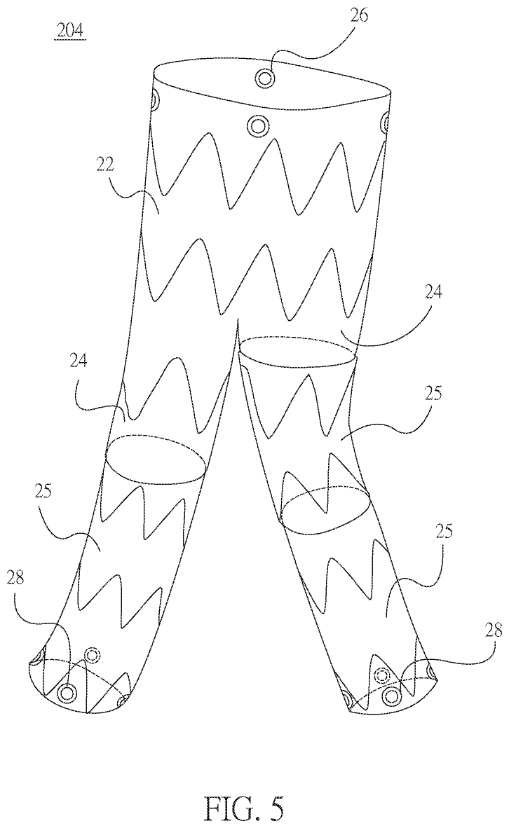

Referring to FIG. 5, a stent 204 in accordance with a fourth preferred embodiment of the invention is shown. The characteristics of the stent 204 of the fourth preferred embodiment are described below. The stent 204 is inserted into a blood vessel and is implemented as including a first stent member 22 inserted into an artificial blood vessel. The first stent member 22 is separated into two second stent members 24 inserted into the artificial blood vessel. A third stent 25 into the artificial blood vessel is extended from the left second stent member 24, another third stent 25 into the artificial blood vessel is extended from the right second stent member 24, and a further third stent 25 into the artificial blood vessel is extended from another third stent 25. A plurality of third magnetic members 28 are spaced around the third stent 25 proximate an open end of the third stent 25 on the left of FIG. 5 and a plurality of third magnetic members 28 are spaced around the further third stent 25 proximate an open end of the further third stent 25.

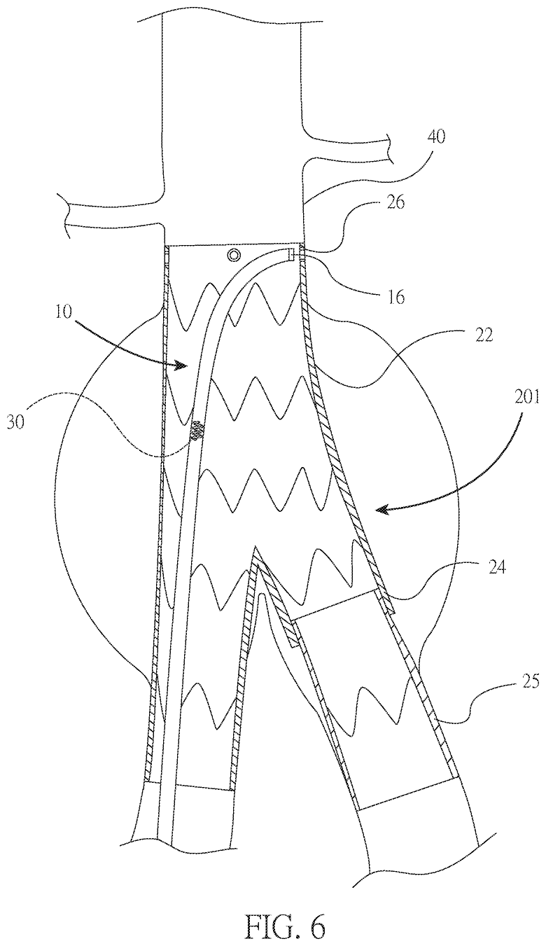

Referring to FIG. 6 in conjunction with FIGS. 1 and 2, the stent 201 is placed in an artery 40 of abdomen. When the positioning member 10 passes through the stent 201, a user may hold the handle 12 to insert the extension 14 through the second stent member 24. The extension 14 further enters the first stent member 22. As described above, the second magnetic members 26 are spaced around an end of the stent 22. Thus, the first magnetic member 16 at the open end of the extension 14 is attracted by the second magnetic members 26. At least one screw (one is shown) 30 is provided in the positioning member 10. The screw 30 is placed from an end of the handle 12 and further moves along an axis of the extension 14 until reaches an end of the first magnetic member 16. The second magnetic members 26 attach to the first magnetic member 16 due to magnetic field. Thus, the screw 30 is fastened in a position proximate the second magnetic members 26. As shown in FIG. 6, the third stent 25 is placed in the second stent member 24.

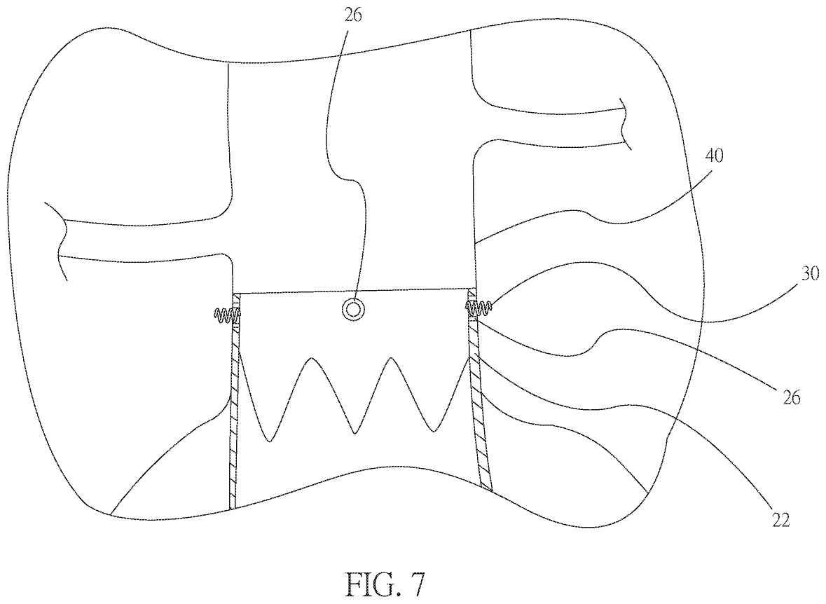

Referring to FIG. 7 in conjunction with FIG. 6, the stent 201 is placed in the artery 40 of the abdomen and the screws 30 are fastened. In detail, each screw 30 is fastened in a central, circular hole of the second magnetic member 26. Taking the stent 202 of FIG. 3 placed in the artery 40 of the abdomen as an example, positions proximate the second magnetic members 26 are defined as predetermined positions to be attached. Thus, each screw 30 is fastened in the predetermined position. As a result, the stent 201 is securely placed in the artery 40 of the abdomen.

While the invention has been described in terms of preferred embodiments, those skilled in the art will recognize that the invention can be practiced with modifications within the spirit and scope of the appended claims.

What is claimed is:

1. A positioning device for a stent, comprising:
a positioning member including a handle and an extension secured to the handle wherein the handle and the extension are coaxially connected, and an open end of the extension is provided with a hollow first magnetic member; and
a stent inserted into a blood vessel and including a first stent member inserted into an artificial blood vessel, the first stent member being separated into two second stent members inserted into the artificial blood vessel wherein a diameter of the first stent member is greater than that of each second stent member, and a diameter of each second stent member is greater than that of the extension, the stent further comprising at least one second magnetic member on the first stent member proximate an open end thereof, the second magnetic member being configured to attach to the first magnetic member.

2. The positioning device for stent of claim 1, further comprising at least one screw in the positioning member wherein the screw is placed from an end of the handle and moves through the extension until reaches a predetermined position of attaching the at least one second magnetic member to the first magnetic member, and the predetermined position is a center of the second magnetic member.

3. The positioning device for stent of claim 1, wherein the number of the at least one second magnetic member is more than one, and the second magnetic members are spaced apart.

4. The positioning device for stent of claim 2, wherein the number of the at least one second magnetic member is more than one, and the second magnetic members are spaced apart.

5. The positioning device for stent of claim 1, further comprising at least one third stent extended from the at least one second stent member into the artificial blood vessel wherein the at least one third stent is disposed in the at least one second stent member.

6. The positioning device for stent of claim 5, further comprising a third stent into the artificial blood vessel extended from a left one of the second stent members, another third stent into the artificial blood vessel extended from a right one of the second stent members, and a further third stent into the artificial blood vessel extended from the another third stent.

7. The positioning device for stent of claim 5, further comprising at least one third magnetic member spaced around one of the at least one third stent proximate an open end thereof, and at least one third magnetic member spaced around another one of the at least one third stent proximate an open end thereof.

8. The positioning device for stent of claim 1, wherein the second magnetic members are hollow circular or solid rectangular.
